(19) United States
(12) Patent Application Publication
(10) Pub. No.: US 2003/0088232 A1
(43) Pub. Date: May 8, 2003

(54) Container cap including a luer connector

(76) Inventor: Todd Duell, San Diego, CA (US)

Correspondence Address: STETINA BRUNDA GARRED & BRUCKER, 75 ENTERPRISE, SUITE 250, ALISO VIEJO, CA, 92656, US

(21) Appl. No.: 10/007,936

(22) Filed: Nov. 8, 2001

(51) Int. Cl. A61M 5/32; A61B 19/00

(52) U.S. Cl. 604/411; 604/414

(57) ABSTRACT

The present invention may be regarded as a container cap for attachment to a secondary container to facilitate the transfer of liquid materials from a device into the secondary container. The container cap includes a cap body configured to attach to the secondary container. The inner surface of the cap body may include screw threads that enable the cap to be attached to the secondary container. The cap body may include a ribbed or rough outer surface used for gripping in order to facilitate attachment of the container cap to the secondary container. The container cap includes a male or female luer connector used for facilitating the transfer of the fluids from the device to the secondary container.

### CROSS-REFERENCE TO RELATED APPLICATIONS

(Not Applicable)

### STATEMENT RE: FEDERALLY SPONSORED RESEARCH/DEVELOPMENT

(Not Applicable)

### BACKGROUND OF THE INVENTION

The present invention is generally related to container caps, and more particularly to a container cap including a luer connector and a vent hole.

Luer fittings or connectors are known in the art. For example, a luer fitting may be used to transport fluids an intravenous (IV) bag to tubing leading to a catheter that is inserted into a blood vessel of a patient. Luer connectors are also used to connect a filtering device to a fluid transport device, such as a syringe. If a fluid needs to be filtered, the luer filter and syringe are typically taped to a secondary container into which the filtered fluid will be transferred. Such containers are not stable are can easily fall apart and/or tip over.

Thus, there is a need for a sterile or non-sterile environment in which to transfer fluids into a secondary container.

### BRIEF SUMMARY OF THE INVENTION

An aspect of the invention may be regarded as a container cap including a luer connector. The container cap includes a cap body configured to fit a secondary container. The cap body includes a top surface, a bottom surface and a side surface having an inner side surface and an outer side surface. There is a luer fitting disposed through the top surface of the cap configured to allow back pressure relief while materials are transferred into the secondary container.

In accordance with other aspects of the invention, the luer fitting may be a male luer fitting or a female luer fitting.

In accordance with still other aspects of the invention, the vent hole is a second luer fitting that is connectable to a secondary device for sterile venting.

In accordance with further aspects of the invention, the outer side surface is a rough surface configured for gripping and handling of the system.

In accordance with still further aspects of the invention, the inner side surface is configured for secure attachment to a secondary container. The inner side surface may be threaded for secure attachment to the secondary container.

### BRIEF DESCRIPTION OF THE DRAWINGS

These as well as other features of the present invention will become more apparent upon reference to the drawings wherein:

FIG. 1 is a top elevation view of a cap for attachment to a secondary container, the cap including a luer connector for connection to a device, and a vent hole;

FIG. 2 is a side elevation view of the container cap of claim 1;

FIG. 3 is a side section view of the container cap of claim 1;

FIG. 4 is a perspective view of the container cap of claim 1;

FIG. 5 is an alternative embodiment of a cap for attachment to a secondary container, the cap including multiple luer connectors for connection to a device and a secondary device, and a vent hole;

FIG. 6 is a side elevation view of the container cap of claim 5;

FIG. 7 is a side section view of the container cap of claim 5; and

FIG. 8 is a perspective view of the container cap of claim 5.

### DETAILED DESCRIPTION OF THE INVENTION

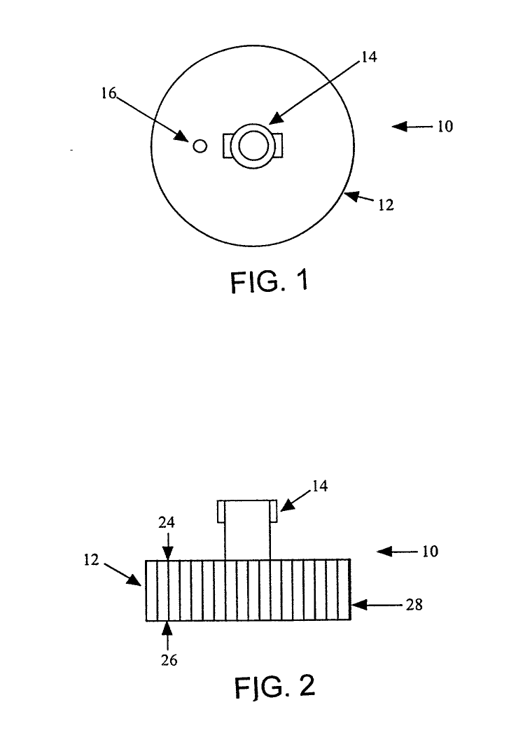

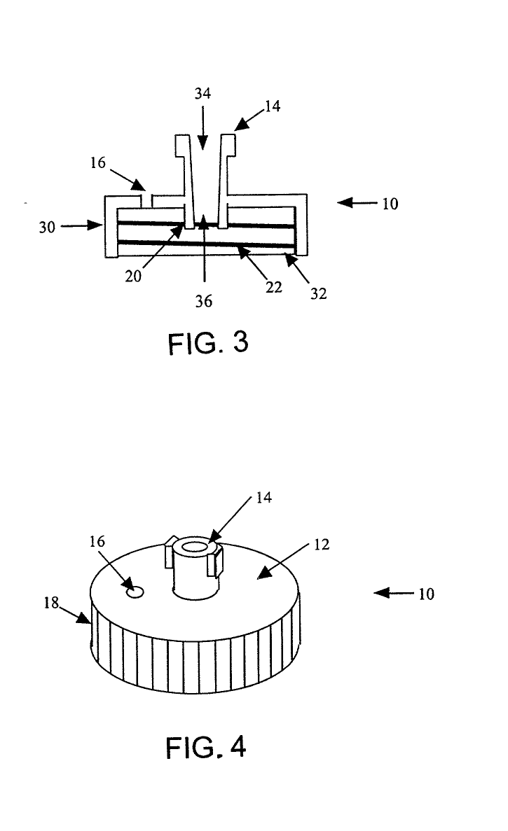

Referring now to the drawings wherein the showings are for purposes of illustrating preferred embodiments of the present invention only, and not for purposes of limiting the same, FIGS. 1-4 illustrate an exemplary embodiment of a container cap 10 including a luer fitting or connector 14 formed in accordance with the present invention. The container cap 10 is placed on a secondary container that receives fluid materials from a device, for example, a fluid filtering device, via the luer connector 14. In exemplary embodiments, the cap 10 includes a cap body 12 that is generally cylindrical in shape and resembles a fitted cap as shown in FIG. 4. It will be appreciated that the size and shape of the cap body 12 vary based on the configuration of the opening of the secondary container to which the cap is going to be attached.

The cap 10 may be manufactured of materials such as polypropylene, polycarbonate, polyethelene, stainless steel, aluminum, or any other alloy metal. The cap body 12 comprises a top surface 24, a bottom surface 26, and a side surface having an inner side surface 30 and an outer side surface 28. As shown in FIG. 2, the outer side surface 28 may be ribbed or hatched to create a rough surface for the purpose of improving the grip and handling of the container cap 10 while attaching the container cap 10 to the secondary container.

The inner side surface 30 is sized and configured to fit the secondary container. The inner side surface 30 may include threads (as shown in FIG. 2) or other camming or alignment mechanisms to attach the cap 10 of the present invention to the secondary container in order to minimize leakage. Because of the universal connection capabilities, the container cap 10 can be configured to connect to a variety of containers by changing the diameter of the cap body 12 and the screw threads necessary to connect to the secondary container.

The top surface 24 of the cap body 12 includes a luer fitting or connector 14. The luer connector 14 may be a male luer connector or a female luer connector to enable a wide variety of devices to connect to the container cap 10 of the present invention by way of a complementary female or male luer fitting. When the container cap 10 is attached to the secondary container, a cap for the luer connector 14 may be fitted to the luer connector 14 to seal the secondary container. Preferably, the luer fitting is located at or near the center of the top surface 24 of the cap body 12. The luer fitting or cone 14 extends from the upper surface 24 of the cap body 12 through to an inner cavity 32 of the cap 10. Thus, the luer cone 14 forms a passage 34 through the cap body 12 ending in the internal cavity 32 in a small aperture 36. The passage 34 may have a protruding tip 20 that projects into the internal cavity 32 of the cap 10 of the present invention as shown in the figures or the tip may end at the cap body 12. The protruding tip may facilitate the smooth transfer of material from the device into the secondary container.

The top surface 24 of the cap body 12 also includes a vent hole 16 that is of sufficient size to allow back pressure relief while materials are transferred into the secondary container. In exemplary embodiments, the vent hole is preferably in the range of one millimeter-three millimeters in diameter.

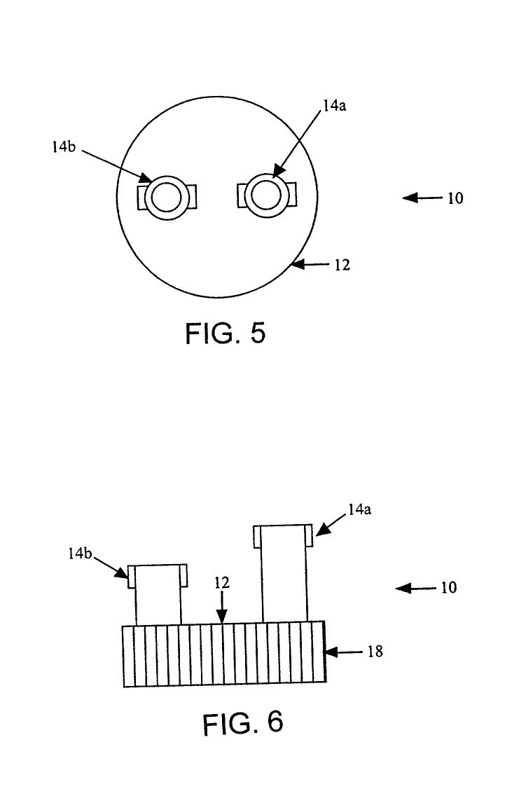

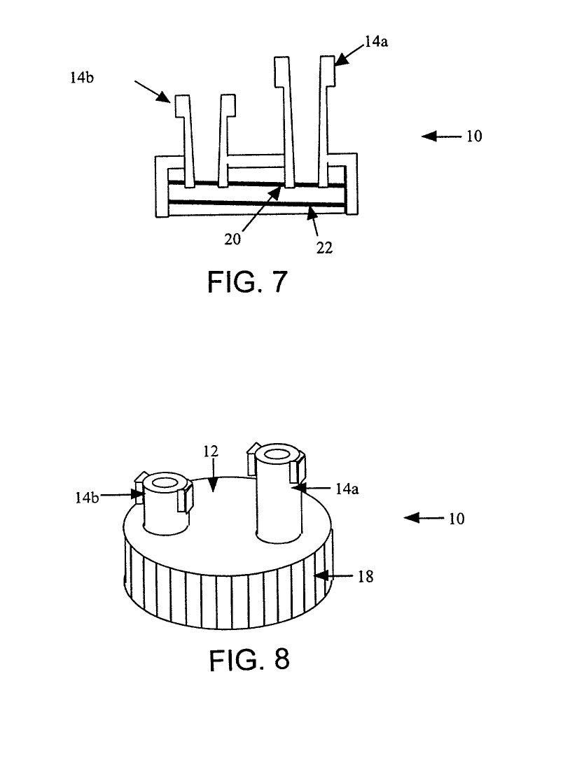

FIGS. 5-8 illustrate an alternative embodiment of the present invention that allows for the connection of an additional sterile venting device. Two luer fittings 14a and 14b extend from the upper surface 24 of the cap body 12 through to the inner cavity 32 of the container cap 10. The luer fittings 14a and 14b may be male and/or female luer fittings. To enable the connection of a sterile filtering device, one of the luer fittings may be longer than the other to allow enough space for the connection of multiple devices. The luer fittings 14a and 14b are also spaced far enough apart to allow sufficient clearance for the attachment of multiple devices. The luer fittings 14a and 14b may also be angled outward to provide additional clearance for the attachment of multiple devices.

A well-known method of manufacturing materials for sterile preparations is the injection molding method in which the cap 10 is molded from thermoplastics which include, but are not limited to, polypropolene, polycarbonate, or polyethelene. This method is a convenient and inexpensive method of manufacturing such materials and has the particular advantage that the entire process may be carried out in a sterile or aseptic environment such that the sterility of the material can be guaranteed. However, the thermoplastics used may be incompatible with the chemicals that may come into contact with them.

Another well known method of manufacturing materials for sterile preparations is the metal fabrication method in which the cap 10 is either forged or caste from metal materials that include, but are not limited to various grades of stainless steel, aluminum or other alloy metals. This method is more expensive than the injection molding method described above, but has the particular advantages that the cap 10 may be sanitized and reused for multiple applications and may also be more resistance to some chemicals which may come into contact with the cap 10.

While an illustrative and presently preferred embodiment of the invention has been described in detail herein, it is to be understood that the inventive concepts may be otherwise variously embodied and employed and that the appended claims are intended to be construed to include such variations except insofar as limited by the prior art.

I claim:

1. A system for transferring fluids from a device to a secondary container, the system comprising:
a cap body configured to fit the secondary container, the cap body including a top surface, a bottom surface and a side surface having an inner side surface and an outer side surface;
a luer fitting disposed through the top surface of the cap body creating a passage which is connectable to the device such that the fluids can be transferred from the device through the passage into the secondary container; and
a vent hole disposed through the top surface of the cap body configured to allow back pressure relief while the fluids are transferred into the secondary container.

2. The system of claim 1, wherein the luer fitting is a male luer fitting.

3. The system of claim 1, wherein the luer fitting is a female luer fitting.

4. The system of claim 1, wherein the vent hole comprises a second luer fitting connectable to a secondary device for sterile venting.

5. The system of claim 1, wherein the outer side surface of the cap body is a rough surface configured for gripping and handling of the system.

6. The system of claim 1, wherein the inner side surface of the cap body is configured for secure attachment to the secondary container.

7. The system of claim 6, wherein the inner side surface is threaded for secure attachment to the secondary container.

8. The system of claim 1, wherein the vent hole is in the range of about one millimeter in diameter to about 3 millimeters in diameter.

9. A method for transferring a fluid material from a device having a luer connector and a passageway into a secondary container, the method comprising:
a) securely attaching a cap comprising a luer connector complementary to the luer connector of the device and a vent hole to the secondary container;
b) attaching the luer connector of the device to the complementary luer connector of the cap; and
c) transferring the fluid from the device to the secondary container via the passageway of the luer connector of the cap.

10. The method of claim 9, wherein the device is a filtering device.

11. The method of claim 9, wherein the luer connector of the cap is a male luer connector and the luer connector of the device is a female luer connector.

12. The method of claim 9, wherein the luer connector of the cap is a female luer connector and the luer connector of the device is a male luer connector.

13. The method of claim 9, wherein the vent hole comprises a second luer connector configured to provide a sterile environment for transferring the fluid from the device to the secondary container via the passageway of the luer connector of the cap.

14. The method of claim 9, wherein the cap includes a threaded inner surface for securely attaching the cap comprising the luer connector complementary to the luer connector of the device and a vent hole to the secondary container.
